(19) United States
(12) Patent Application Publication
(10) Pub. No.: US 2026/0171198 A1
(43) Pub. Date: Jun. 18, 2026

(54) SYSTEM FOR RECRUITING CLINICAL TRIAL TARGETS BASED ON BIG DATA

(71) Applicant: GREEN-RIBBON Corp., Daejeon (KR)

(72) Inventor: Gyu Ri KIM, Daegu (KR)

(73) Assignee: GREEN-RIBBON Corp., Daejeon (KR)

(21) Appl. No.: 19/068,476

(22) Filed: Mar. 3, 2025

(30) Foreign Application Priority Data
Dec. 12, 2024 (KR) 10-2024-0184668

(51) Int. Cl. G16H 10/20; G06F 21/31; G06F 21/62; G06F 21/64; G06Q 20/10; G16H 15/00; G16H 40/67; G16H 50/30

(52) U.S. Cl. CPC G16H 10/20; G06F 21/31; G06F 21/6245; G06F 21/64; G06Q 20/102; G16H 15/00; G16H 40/67; G16H 50/30

(57) ABSTRACT

Provided is a system for recruiting clinical trial targets on the basis of big data, the system including a user terminal configured to upload medical data to inquire about available insurance funds and receive a list of clinical trials matching the medical data, at least one research institution terminal configured to register clinical trial requirements and request recruitment of clinical trial targets, and a management service provision server including a reception unit configured to receive the request from the at least one research institution terminal, a matching determination unit configured to determine whether the medical data uploaded by the user terminal matches the clinical trial requirements, and a transmission unit configured to transmit a list of clinical trials matching the medical data to the user terminal.

### CROSS-REFERENCE TO RELATED APPLICATION

This application claims priority to and the benefit of Korean Patent Application No. 10-2024-0184668, filed on Dec. 12, 2024, the disclosure of which is incorporated herein by reference in its entirety.

### BACKGROUND

### 1. Field of the Invention

The present invention relates to a system for recruiting clinical trial targets on the basis of big data, and more particularly, to a system for recruiting clinical trial targets rapidly and accurately by extracting a list of clinical trials with matching medical data and proposing participation.

### 2. Discussion of Related Art

New drug development, which represents the core of the pharmaceutical industry, is considered a major future growth industry because high added-value is created along with a high investment risk caused by requirements of a long period of time, great costs, and a low probability of success. A new drug development process is divided into a discovery stage of a therapeutic agent and a development stage, and the most important part of the development stage is the clinical pharmacology and clinical trials for therapeutic exploration and confirmation. In Korea, the market for clinical trials has increased quantitatively under the government's leadership, and for sponsors who commission clinical trials and for investigators who plan and conduct investigator-initiated trials, recruiting and enrolling an adequate number of clinical trial subjects is the most difficult and most important step in the process of conducting clinical trials. Since a delay in a subject recruitment period may result in a delay in the approval of a new drug, it is very important to thoroughly manage this step. Accordingly, clinical trial providers, such as a sponsors and researchers, develop strategies to recruit clinical trial subjects efficiently and rapidly.

Platforms for recruiting and managing clinical trial participants have been researched and developed. In this regard, Korean Patent Registration No. 10-1510600 (published on Apr. 8, 2015) and Korean Patent Application Publication No. 2019-0094795 (published on Aug. 14, 2019) disclose a platform that checks an intention to participate in a clinical trial from a user terminal when health data is registered and offers users who have agreed to participation to participate in clinical trials when there is a demand for the clinical trials and a platform that provides information required for performing clinical trials to user terminals of clinical trial subjects and manages the users during clinical trial periods.

The former uses the health data of users, but most clinical trials utilize medical data rather than health data. Accordingly, even when users are given an offer to participate, the users are likely to be rejected because the users do not have matching requirements for a clinical trial. The latter is not for recruiting clinical trial targets but only discloses a management method thereon. Recently, research organizations have surveyed pharmaceutical companies and found that clinical delays are the most critical issue with respect to clinical data release. To produce clinical results, it is necessary to complete clinical trials, and to complete clinical trials, it is necessary to enroll all subjects. However, more than 75% of clinical trials fail to enroll subjects within a determined period. Consequently, it is necessary to research and develop a recruiting platform for rapidly enrolling clinical trial subjects.

### SUMMARY OF THE INVENTION

The present invention is directed to providing a system for recruiting clinical trial targets on the basis of big data that stores medical data mapped to the user terminal when the medical data is uploaded from a user terminal to inquire about available insurance funds, extracts user terminals having the medical data corresponding to clinical trial requirements when a research institution terminal requests target recruiting while registering the clinical trial requirements, proposes participation in the clinical trial while transmitting a list of matching clinical trials to the user terminal, and thereby helps recruit subjects rapidly and accurately and complete clinical trials without delays. However, technical objects to be achieved by the present embodiment are not limited to those described above, and other technical objects may exist.

According to an aspect of the present invention, there is provided a system for recruiting clinical trial targets on the basis of big data, the system including a user terminal configured to upload medical data to inquire about available insurance funds and receive a list of clinical trials matching the medical data, at least one research institution terminal configured to register clinical trial requirements and request recruitment of clinical trial targets, and a management service provision server including a reception unit configured to receive the request from the at least one research institution terminal, a matching determination unit configured to determine whether the medical data uploaded by the user terminal matches the clinical trial requirements, and a transmission unit configured to transmit a list of clinical trials matching the medical data to the user terminal.

### BRIEF DESCRIPTION OF THE DRAWINGS

The above matters and other objects, features, and advantages of the present invention will become more apparent to those of ordinary skill in the art by describing exemplary embodiments thereof in detail with reference to the accompanying drawings, in which:

FIG. 1 is a diagram illustrating a system for recruiting clinical trial targets on the basis of big data according to an exemplary embodiment of the present invention;

FIG. 2 is a block diagram of a management service provision server included in the system of FIG. 1;

FIGS. 3A-3M and 4A-4I are diagrams illustrating an example of implementing a clinical trial target recruitment management service according to the exemplary embodiment of the present invention; and

FIG. 5 is an operational flowchart illustrating a method of providing the clinical trial target recruitment management service according to the exemplary embodiment of the present invention.

### DETAILED DESCRIPTION OF EXEMPLARY EMBODIMENTS

Hereinafter, exemplary embodiments of the present invention will be described in detail such that those skilled in the technical field to which the present invention pertains can readily implement the present invention upon referencing the accompanying drawings. However, the present invention can be implemented in a variety of different forms and is not limited to the embodiments described herein. In the drawings, parts irrelevant to the description will be omitted to clearly describe the present invention, and similar reference numerals will be assigned to similar components throughout the specification.

Throughout the specification, when a part is referred to as being “connected to” another part, the part may be “directly connected” to the other part and may also be “electrically connected” to the other part with still another element intervening therebetween. In addition, when a part is referred to as “including” a component, this implies the inclusion of other components rather than the exclusion of any other components unless particularly described otherwise and should be understood as not precluding the possibility of presence or addition of one or more other features, integers, steps, operations, components, parts, or combinations thereof.

The terms “about,” “substantially,” and the like used throughout the specification mean the values or values close thereto when manufacturing and material tolerances specific to the stated meaning are presented and are used to prevent unconscionable abusers from unfairly using the disclosure of values precisely or absolutely described to aid the understanding of the present invention. The term “operation” or “operation of” used throughout the present specification of the present invention does not mean “operation for.”

In the present specification, the term “unit” includes a unit implemented by hardware, a unit implemented by software, and a unit implemented by both. Further, one unit may be implemented by two or more pieces of hardware, and two or more units may be implemented by one piece of hardware. Meanwhile, a “unit” is not limited to software or hardware and may be configured to reside in an addressable storage medium or configured to reproduce one or more processors. Therefore, as an example, a “unit” includes components, such as software components, object-oriented software components, class components, and task components, processes, functions, attributes, procedures, subroutines, segments of a program code, drivers, firmware, microcode, circuits, data, databases, data structures, tablets, arrays, and variables. Components and functions provided in “units” may be combined into a smaller number of components and subdivided into additional components and “units”. Further, components and “units” may be implemented to reproduce one or more central processing units (CPUs) in a device or a security multimedia card.

In the present specification, some operations or functions described as performed by a terminal, an apparatus, or a device may be performed instead in a server connected to the terminal, apparatus, or device. Similarly, some operations or functions described as being performed by a server may be performed in a terminal, an apparatus, or a device connected to the server.

In the present specification, some operations or functions described as “mapped to” or “matched to” a terminal may be interpreted as being mapped or matched to a unique number of a terminal or personal identification information, which is identification data of the terminal.

Hereinafter, the present invention will be described in detail with reference to the accompanying drawings.

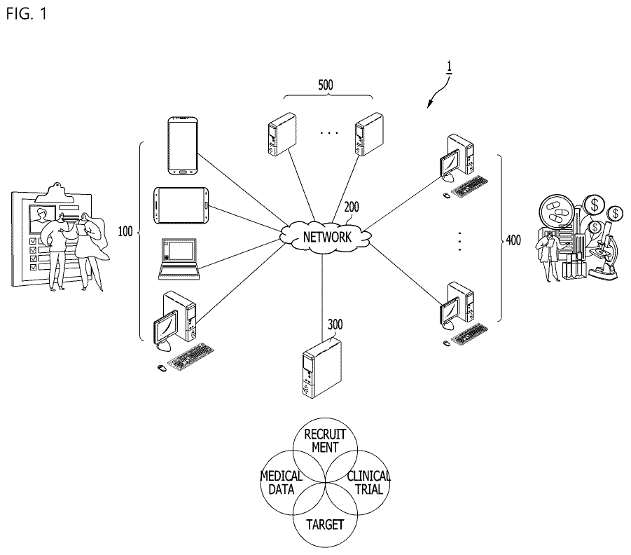

FIG. 1 is a diagram illustrating a system for recruiting clinical trial targets on the basis of big data according to an exemplary embodiment of the present invention. Referring to FIG. 1, a system 1 for recruiting clinical trial targets on the basis of big data may include at least one user terminal 100, a management service provision server 300, at least one research institution terminal 400, and at least one information provision server 500. However, the system 1 for recruiting clinical trial targets on the basis of big data shown in FIG. 1 is merely an exemplary embodiment of the present invention, and thus the present invention is not limited to FIG. 1.

The components of FIG. 1 are generally connected via a network 200. For example, as shown in FIG. 1, the at least one user terminal 100 may be connected to the management service provision server 300 via the network 200. Via the network 200, the management service provision server 300 may be connected to the at least one user terminal 100, the at least one research institution terminal 400, and the at least one information provision server 500. Via the network 200, the at least one research institution terminal 400 may be connected to the management service provision server 300. Via the network 200, the at least one information provision server 500 may be connected to the at least one user terminal 100, the management service provision server 300, and the at least one research institution terminal 400.

The network 200 is a connective structure that allows information to be exchanged between individual nodes such as multiple terminals and servers. Examples of the network 200 include a local area network (LAN), a wide area network (WAN), the Internet (the world wide web (WWW)), a wired or wireless data communication network, a telephone network, a wired or wireless television communication network, etc. Examples of the wireless data communication network include, but are not limited to, a 3rd Generation (3G) network, a 4th Generation (4G) network, a 5th Generation (5G) network, a 3rd Generation Partnership Project (3GPP) network, a 5th Generation Partnership Project (5GPP) network, a 5G New Radio (NR) network, a 6th Generation of cellular networks (6G) network, a Long Term Evolution (LTE) network, a World Interoperability for Microwave Access (WiMAX) network, a Wi-Fi network, the Internet, a LAN, a WAN, a personal area network (PAN), a radio frequency (RF) network, a Bluetooth network, a near-field communication (NFC) network, a satellite broadcast network, an analog broadcast network, a digital multimedia broadcasting (DMB) network, etc.

In the following description, the term “at least one” is defined as a term including the singular and plural meaning. Even when the term “at least one” is not present, each component may be present in a singular or plural form, and it is obvious that the term may have a singular or plural meaning. In addition, the fact that each component is provided in a singular or plural form means it is changeable in accordance with embodiments.

The at least one user terminal 100 may be a terminal of a user who uploads medical data to inquire about available insurance funds and receives a list of clinical trials matching the medical data by using a webpage, an app page, a program, or an application related to a clinical trial target recruitment management service.

Here, the at least one user terminal 100 may be implemented as a computer that may access a server or a terminal at a remote location via a network. The computer may include, for example, a navigation device, a notebook, a desktop, a laptop, etc. equipped with a web browser. In this case, the at least one user terminal 100 may be implemented as a terminal that may access a server or a terminal at a remote location via a network. The at least one user terminal 100 is, for example, a mobile communication device in which portability and mobility are ensured and may be any type of handheld wireless communication device, such as a Personal Communication System (PCS) terminal, a Global System for Mobile Communications (GSM) terminal, a Personal Digital Cellular (PDC) terminal, a Personal Handyphone System (PHS) terminal, a Personal Digital Assistant (PDA) terminal, an International Mobile Telecommunication (IMT)-2000 terminal, a Code Division Multiple Access (CDMA)-2000 terminal, a wideband CDMA (W-CDMA) terminal, a Wireless Broadband Internet (WiBro) terminal, a smartphone, a smartpad, a tablet personal computer (PC), etc.

The management service provision server 300 may be a server that provides the webpage, the app page, the program, or the application related to the clinical trial target recruitment management service. The management service provision server 300 may be a server that performs target recruiting while clinical trial requirements are registered therein by the research institution terminal 400. The management service provision server 300 may be a server that receives medical data uploaded by the user terminal 100, obtains a user satisfying the clinical trial requirements, and then proposes participation in clinical trials while transmitting a list of matching clinical trials to the user terminal 100 of the obtained user. In addition, the management service provision server 300 may be a server that receives an electronic signature for access and use of personal information to access medical history data when there is application for participation from the user terminal 100, collects questionnaire response data when a response to an online medical questionnaire is given on the user terminal 100, and then transmits the collected questionnaire response data to the research institution terminal 400, or obtains users satisfying clinical trial requirements set on the research institution terminal 400 and transmits the users as a subject list to the research institution terminal 400.

The management service provision server 300 may be implemented as a computer that may access a server or a terminal at a remote location via a network. For example, the computer may be a navigation device, a notebook, a desktop, a laptop, etc. equipped with a web browser.

The at least one research institute terminal 400 may be a terminal of a research institute that makes a request to the management service provision server 300 to recruit targets of clinical trials using or without using the webpage, the app page, the program, or the application related to the clinical trial target recruitment management service. To this end, the research institution terminal 400 may be a terminal that registers clinical trial requirements, receives a subject list from the management service provision server 300, and outputs the subject list.

Here, the at least one research institution terminal 400 may be implemented as a computer that may access a server or a terminal at a remote location via a network. For example, the computer may be a navigation device, a notebook, a desktop, a laptop, etc. equipped with a web browser. Alternatively, the at least one research institution terminal 400 may be implemented as a terminal that may access a server or a terminal at a remote location via a network. For example, the at least one research institution terminal 400 is a mobile communication device in which portability and mobility are ensured and may be any type of handheld wireless communication device, such as a PCS terminal, a GSM terminal, a PDC terminal, a PHS terminal, a PDA terminal, an IMT-2000 terminal, a CDMA-2000 terminal, a W-CDMA terminal, a WiBro terminal, a smartphone, a smartpad, a tablet PC, etc.

The at least one information provision server 500 may be a server that enables access to the medical history data of the user terminal 100 using or without using the webpage, the app page, the program, or the application related to the clinical trial target recruitment management service.

Here, the at least one information provision server 500 may be implemented as a computer that may access a server or a terminal at a remote location via a network. For example, the computer may be a navigation device, a notebook, a desktop, a laptop, etc. with a web browser. Alternatively, the at least one information provision server 500 may be implemented as a terminal that may access a server or a terminal at a remote location via a network. For example, the at least one information provision server 500 is a mobile communication device in which portability and mobility are ensured and may be any type of handheld wireless communication device, such as a PCS terminal, a GSM terminal, a PDC terminal, a PHS terminal, a PDA terminal, an IMT-2000 terminal, a CDMA-2000 terminal, a W-CDMA terminal, a WiBro terminal, a smartphone, a smartpad, a tablet PC, etc.

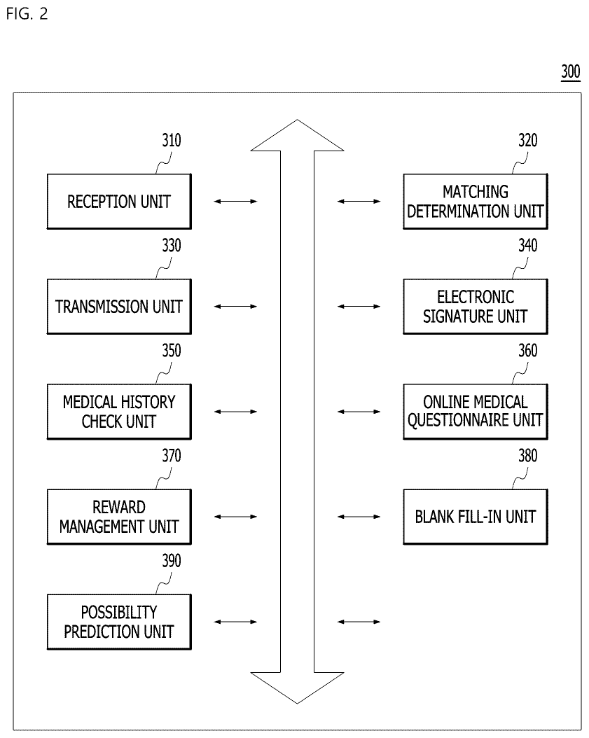

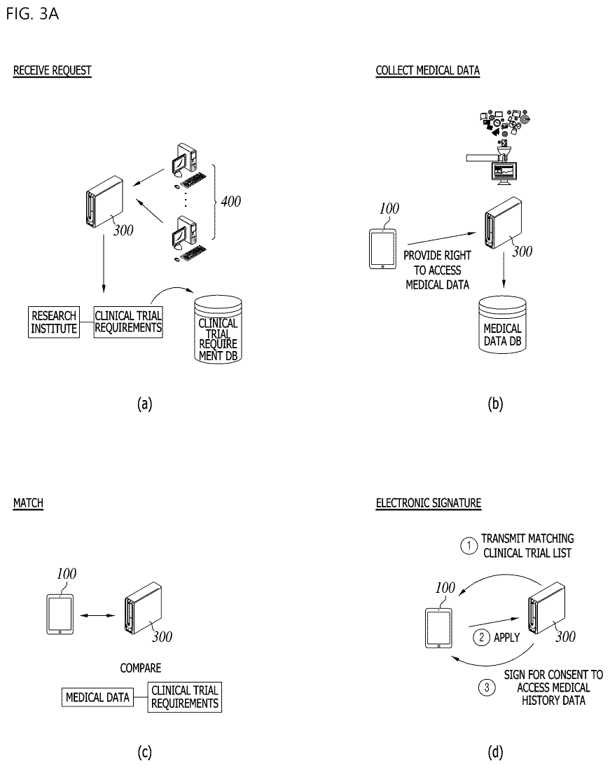

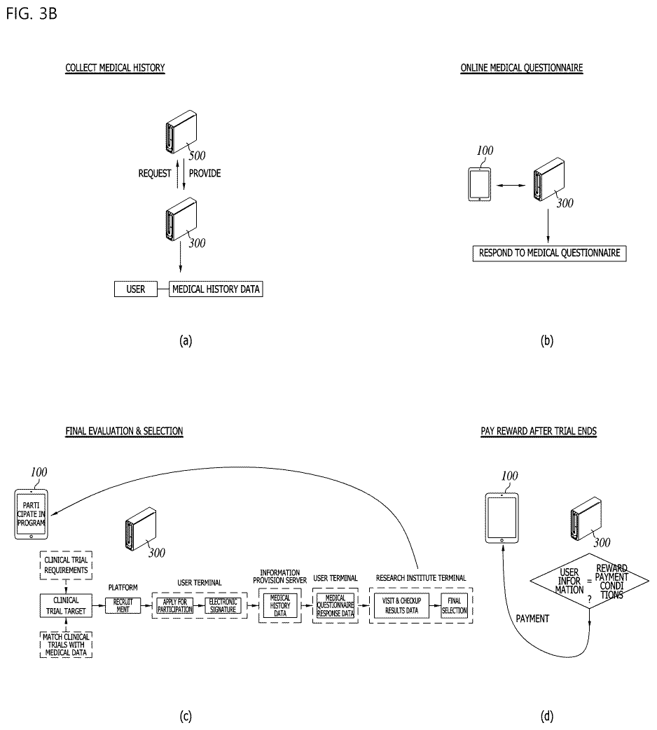

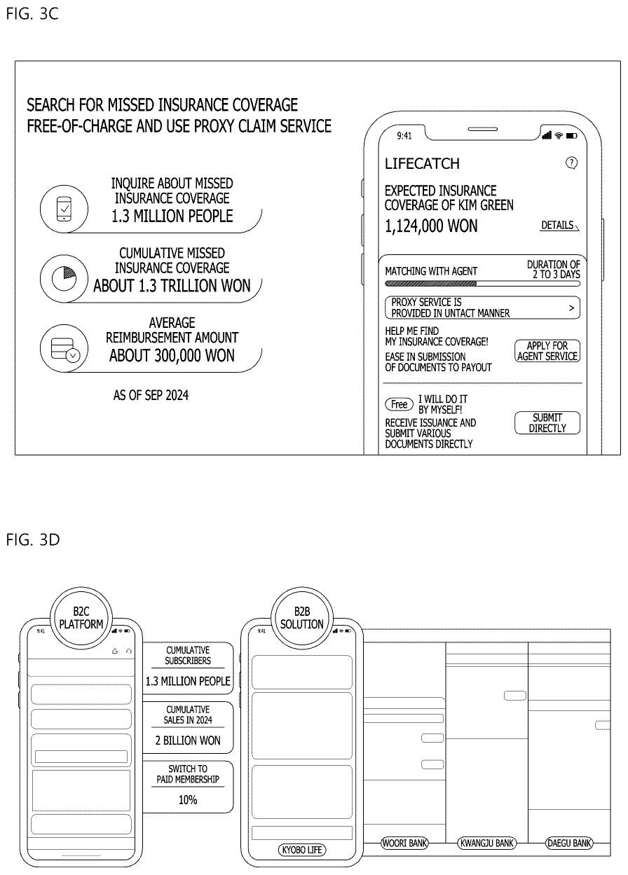

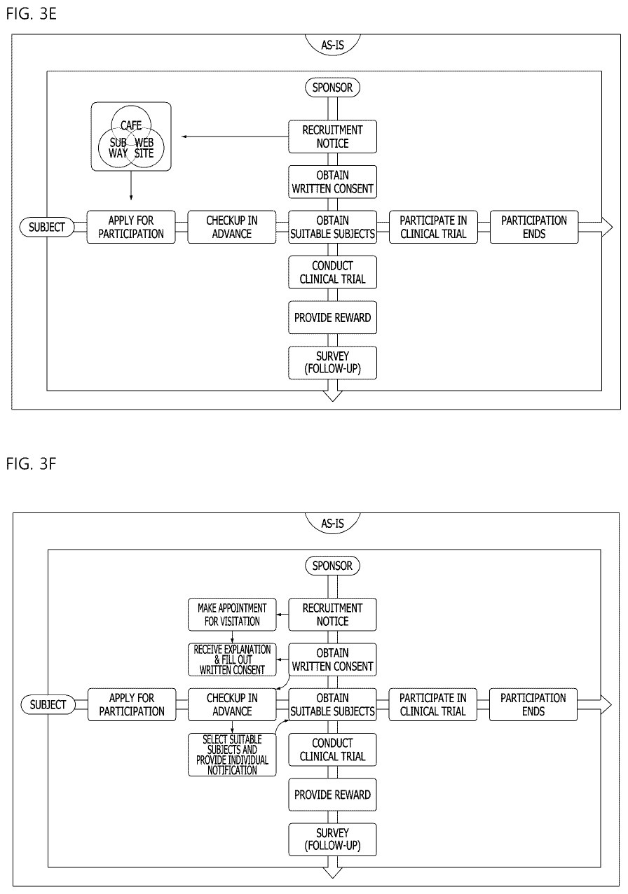

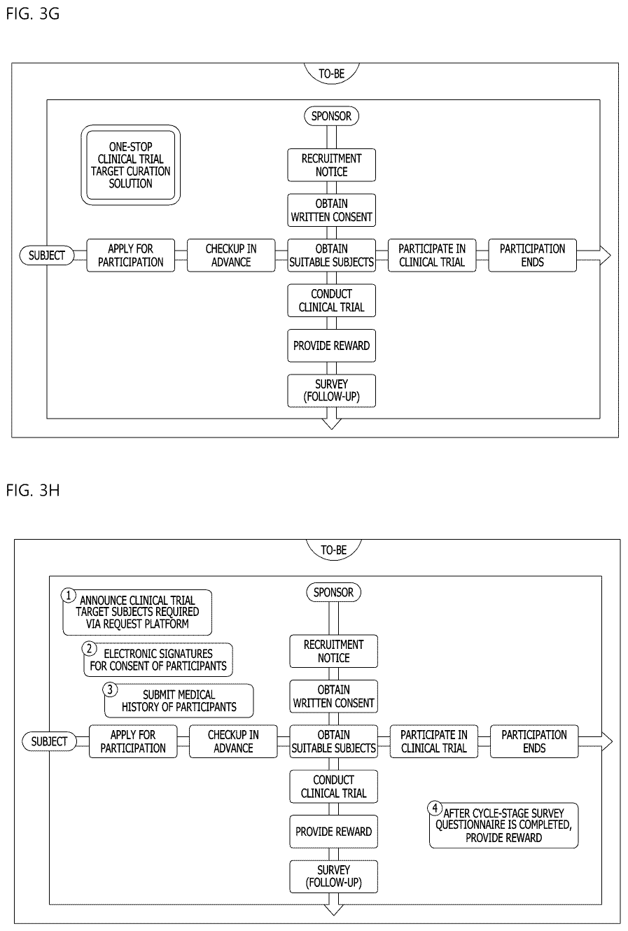

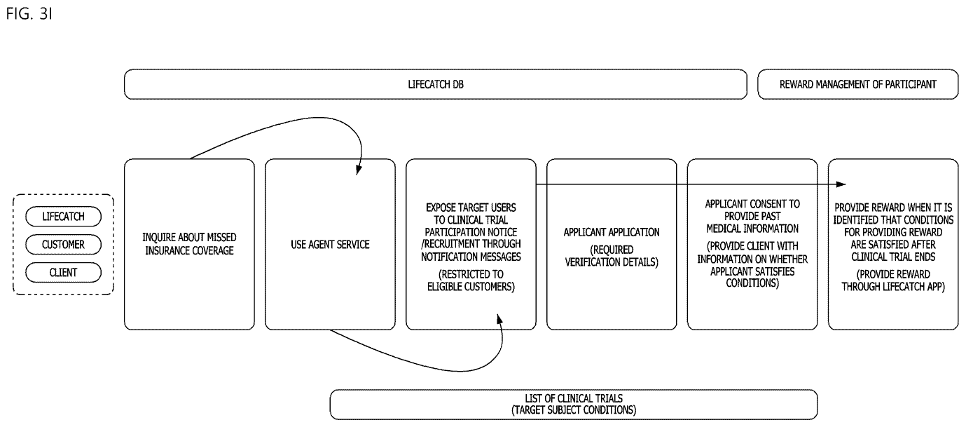

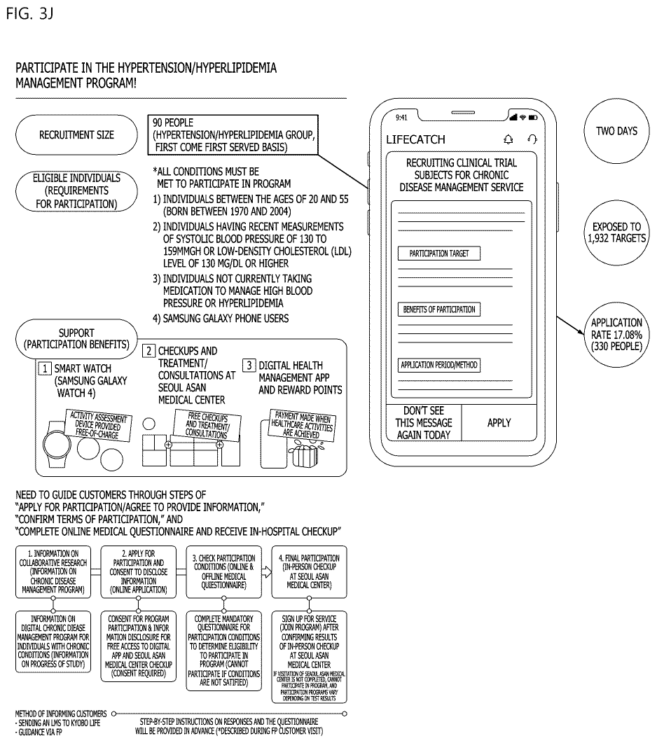

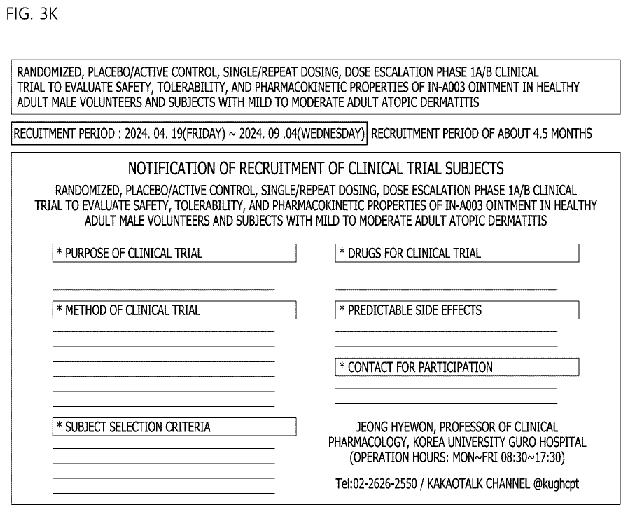

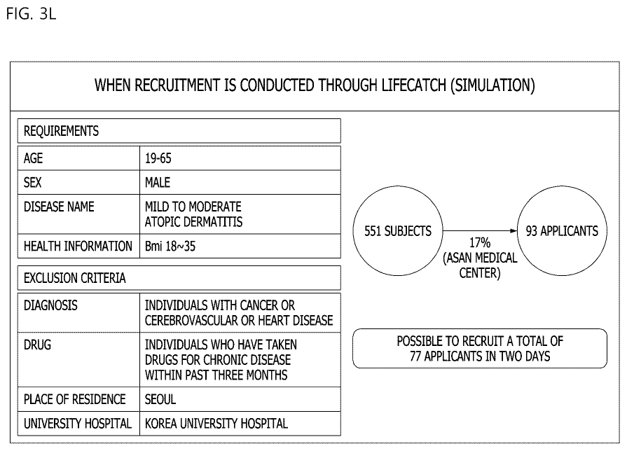

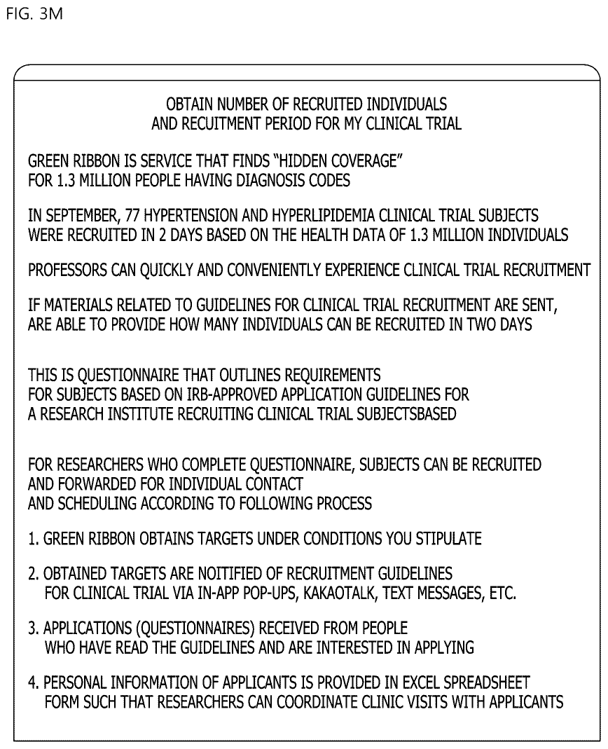

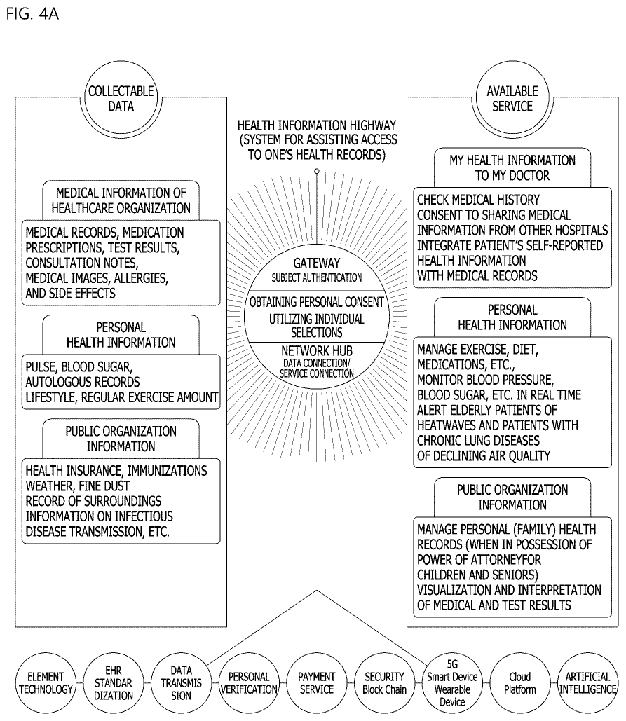

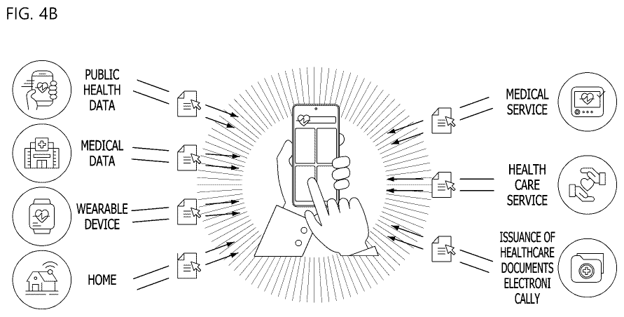

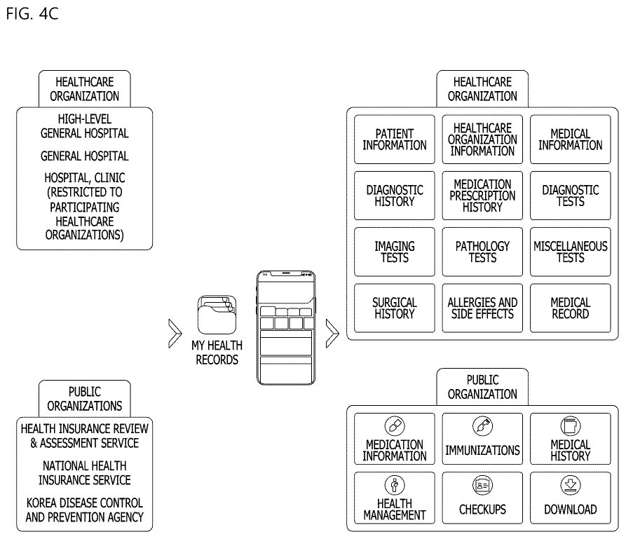

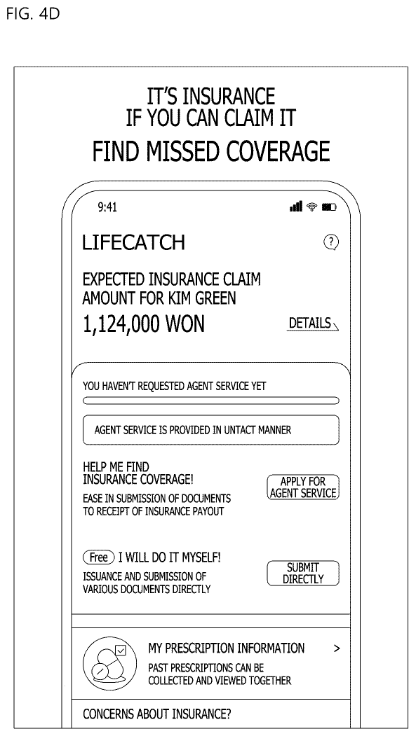

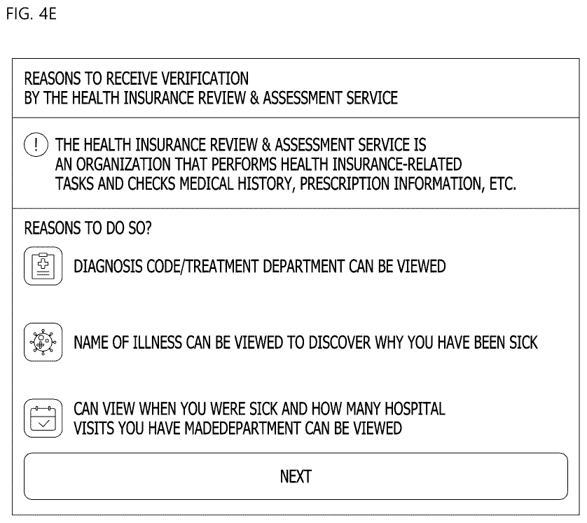

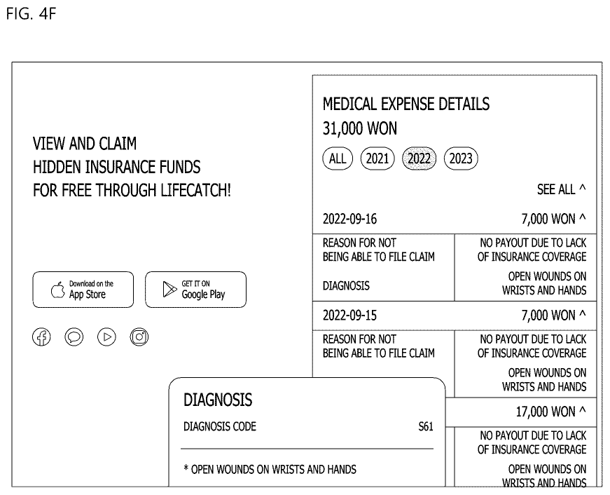

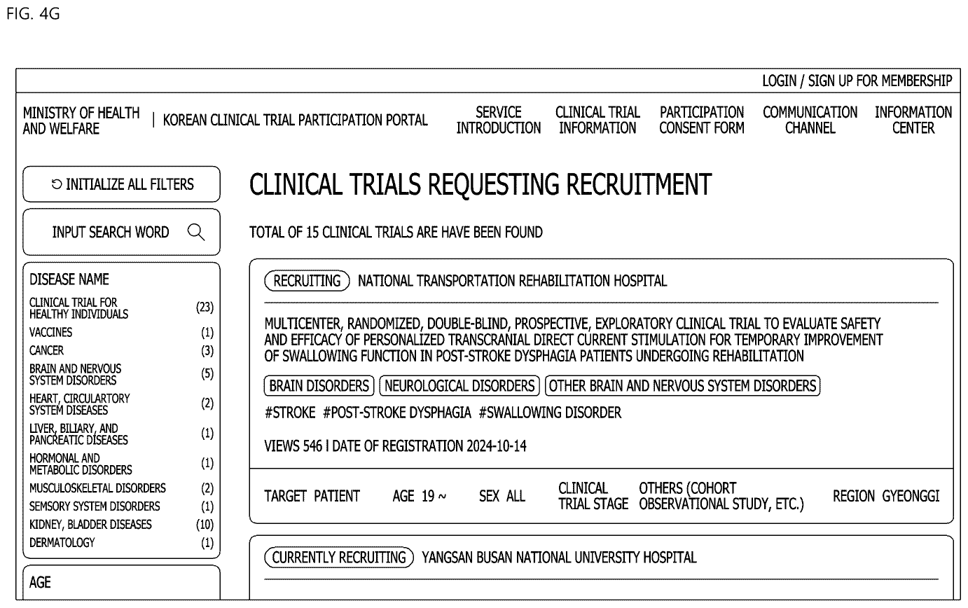

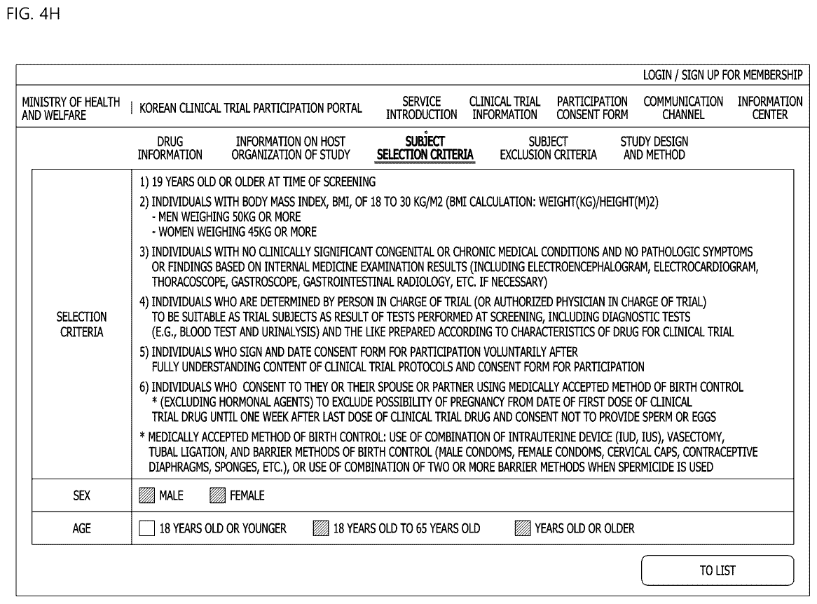

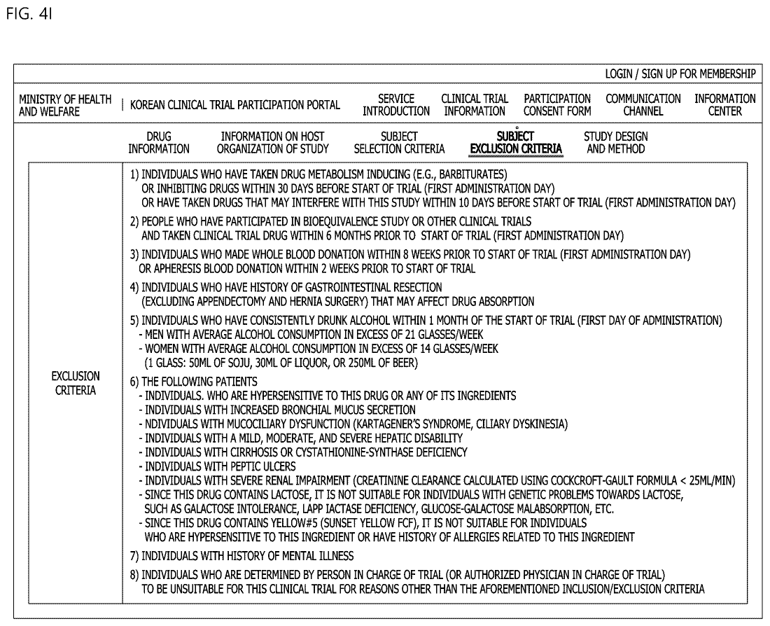

FIG. 2 is a block diagram of a management service provision server included in the system of FIG. 1, and FIGS. 3A-3M and 4A-4I are diagrams illustrating an example of implementing a clinical trial target recruitment management service according to the exemplary embodiment of the present invention.

Referring to FIG. 2, the management service provision server 300 may include a reception unit 310, a matching determination unit 320, a transmission unit 330, an electronic signature unit 340, a medical history check unit 350, an online medical questionnaire unit 360, a reward management unit 370, a blank fill-in unit 380, and a possibility prediction unit 390.

When the management service provision server 300 according to the exemplary embodiment of the present invention or another server (not shown) interoperating with the management service provision server 300 transmits the application, the program, the app page, the webpage, etc. related to the clinical trial target recruitment management service to the at least one user terminal 100 and the at least research institute terminal 400, the at least one user terminal 100 and the at least research institute terminal 400 may install or open the application, the program, the app page, the webpage, etc. related to the clinical trial target recruitment management service. In addition, a script executed by a web browser may be used to run the service program on the at least one user terminal 100 and the at least one research institute terminal 400. Here, the web browser is a program that enables a user to use a web (WWW) service, that is, a program that receives and shows hypertext described in hypertext markup language (HTML). For example, the web browser is Chrome, Microsoft Edge, Safari, FireFox, Whale, UC browser, etc. In addition, the application is an application program on a terminal and may be, for example, an app executed on a mobile terminal (smartphone).

Referring to FIG. 2, the reception unit 310 may receive a request from the at least one research institute terminal 400. The at least one research institute terminal 400 may register clinical trial requirements and the like and request recruitment of clinical trial targets. Here, a clinical trial recruitment notice follows governmental guidelines and is formatted to include items of clinical trial title, purpose, subject selection criteria, clinical trial method, predictable side effects, and incentive related to participation. When a uniform resource locator (URL) or document of the clinical trial recruitment notice is shared by the research institution terminal 400, the reception unit 310 may extract each item from the URL or document and then extract “subject selection criteria” from the items and register the subject selection criteria as clinical trial requirements. For example, table 1 below shows a recruitment notice for a clinical trial currently being conducted at the Gangdong Kyung Hee University Oriental Medicine Hospital.

TABLE 1

| | |
|---|---|
| Sponsoring | Gangdong Kyung Hee University |
| organization | Oriental Medicine Hospital |
| Implementing | |
| organization | |
| Title of clinical | Randomized, controlled, and comparative clinical |
| trial | trial for evaluation of efficacy, safety, and cost- |
| | effectiveness of embedded needle for |
| | temporomandibular joint disorder |
| Purpose of clinical | This is a study to evaluate the efficacy, safety, and |
| trial | cost-effectiveness of embedded needles in patients |
| | with temporomandibular joint disorders who are at |
| | least three months post-onset, and when agreeing |
| | to participate in the study, assignment to a |
| | treatment group or a control group may be |
| | randomly made. |
| Target | 19 years old or older and 70 years old or younger |
| | Patients with temporomandibular joint pain |
| | lasting three months or longer who satisfy |
| | inclusion/exclusion criteria |
| | Individuals who have not started new medication |
| | or stopped taking an existing medication in the past |
| | month |
| Research period | From Sep. 1, 2024 to Dec. 31, 2025 |
| Research period of | Total of 8 visits (6 treatment sessions + 2 follow- |
| clinical trial method | up sessions) |
| | 10 weeks total (6 weeks of treatment + 4 weeks |
| | of follow-up) |
| | Participants will be randomly placed in treatment |
| | and control groups and receive a total of 6 |
| | treatment sessions |
| | Experimental group: embedded acupuncture |
| | treatment once a week |
| | Control group: general physical treatment once a |
| | week |
| Possible adverse | Embedded acupuncture treatment: bruising at the |
| effects | treatment site, pain, foreign body sensation, |
| | swelling, erythema, elevated body temperature, |
| | body aches, and dizziness |
| Incentives provided | Checkup and treatment costs incurred in the |
| to participants | clinical trial |
| | Small transportation fee per visit |

Here, three conditions of “targets” corresponding to “subject selection criteria” may be extracted as clinical trial requirements. Since most clinical trials require visits of subjects, the clinical trial requirements may include the distance from the research institution, that is, the distance from the Gangdong Kyung Hee University Oriental Medicine Hospital provided in the above recruitment notice. Although not sponsored by a research institution, clinical trial recruitment notices registered on the Korean clinical trial participation portal provided by the Ministry of Health and Welfare may also be collected for matching purposes as shown in FIG. 4G.

The matching determination unit 320 may determine whether medical data uploaded by the user terminal 100 matches with the clinical trial requirements. The user terminal 100 may upload medical data to inquire about available insurance funds. Here, the medical data is my medical data shown in FIGS. 4A to 4C. A company (LifeCatch) of the applicant provides a service that extracts a disease code or a disease name using medical data as shown in FIG. 4F, determines whether the disease code or the disease name is a disease covered by an insurance which a user has subscribed to, predicts the amount of insurance payout that may be claimed when the disease is covered by the insurance, and files the insurance claim with each insurance company when the user approves the insurance claim. Accordingly, when a user of each user terminal 100 provides the company of the applicant with a right to access medical data to check his or her available insurance funds, the medical data is mapped to the user terminal 100 and stored, and it is possible to determine whether the medical data matches preregistered clinical trial requirements as shown in Table 1 above.

The transmission unit 330 may transmit a list of clinical trials matching the medical data to the user terminal 100. The user terminal 100 may receive a list of clinical trials matching the medical data. For example, when the user has conditions A, B, C, and D, the user may receive a list of clinical trials in which conditions A, B, C, and D pertain to clinical trial requirements. As shown in FIGS. 3G and 3H, a list of clinical trials is firstly transmitted only to those who are identified as satisfying clinical trial requirements on the basis of medical history (medical codes, diagnostic names, etc.) received through the platform (LifeCatch) of the applicant from the Health Insurance Review & Assessment Service, allowing targeted marketing which is completely different from randomly distributing advertisements in subways, cafes, and websites as shown in FIG. 3E.

The electronic signature unit 340 may provide an electronic signature link to the user terminal 100 to obtain written consent to apply for participation when there is application for participation from the user terminal 100 in at least one clinical trial in the clinical trial list and may map an electronic signature of the user to the user terminal 100 and store the electronic signature when the electronic signature is completed at the user terminal 100. A written consent process shown in FIG. 3E is replaced with an electronic signature as shown in FIG. 3H.

After the consent is received, the medical history check unit 350 may collect medical history data of the user terminal 100 from the at least one information provision server 500. Accordingly, a process of obtaining written consent and then selecting appropriate subjects through a pre-checkup as shown in FIG. 3F may be replaced with a process of directly collecting medical history data required by the research institution from the information provision server 500 and submitting the collected medical history data to the research institute terminal 400 on the basis of electronic consent as shown in FIG. 3H, which can reduce the consumption of temporal and human resources. In this way, it is secondly determined whether a user is a subject satisfying the clinical trial requirements. Referring to FIG. 3I, users who fail to qualify during this operation do not receive an online questionnaire pertaining to the next operation.

After the consent is received, the online medical questionnaire unit 360 may provide the user terminal 100 with an online questionnaire to determine whether the user satisfies the clinical trial requirements. Using a method of ultimately asking the user whether he or she satisfies the clinical trial requirements and receiving the response effectively replaces the pre-checkup process. In this way, it is thirdly determined whether the user is a subject satisfying the clinical trial requirements. Referring to FIG. 3I, users who fail to qualify during this operation are not included in a suitable subject list and do not continue on in the process any longer. As shown in FIG. 3B (c), when users are selected as subjects satisfying the clinical trial requirements up to this operation, the platform of the present invention transmits the subject list to the research institute terminal 400, the users visit the research institute and receive a checkup, and then it is finally determined whether the users are selected or rejected. Finally, selected users participate in the clinical trial over a preset clinical trial period.

Here, the online medical questionnaire unit 360 may provide an online questionnaire using generative artificial intelligence (AI) such as in a format in which a human asks a question and the generative AI gives an answer. For example, referring to the subject selection criteria of FIG. 4H, many people do not know their own body mass indexes (BMIs) which are an index of obesity, and thus starting with BMI, which is the second criterion, the online questionnaire may be provided by the generative AI asking users about their height and weight and checking the degree of obesity. In addition, referring to the subject exclusion criteria in FIG. 4I, it is checked whether a user drinks 21 glasses of an alcoholic beverage a week on average. However, when the user does not know how many bottles correspond to 21 glasses, he or she cannot give an answer as to whether he or she drinks 3 bottles (seven glasses per bottle). Accordingly, when the user asks how many bottles correspond to 21 glasses, the generative AI answers the question, and then the user identifies whether he or she drinks 3 bottles, thus preventing the online questionnaire from being stopped.

The question-and-answer process may be structured in the same format as a doctor actually asking a patient about his or her condition, and the generative AI may learn the process thereof. Through this process, it is possible to provide input data for a disease prediction algorithm of the possibility prediction unit 390, which will be described below, and help the possibility prediction unit 390 with disease prediction. For example, provision of medical or health checkup records, such as medical data, is allowed for five to ten years, and thus the system may discover medical history prior to that point in time when the user reveals his or her medical history. All of this history may be inquired of by the generative AI and answered by the user, making it possible to comprehend what medical history or illnesses the user has.

Since family medical history may not be properly grasped based on medical data, the generative AI may be used to family medical history. For example, if the user's maternal grandmother has had a cholecystectomy, the user's uncle has stomach cancer, and the user himself or herself has had stomach bleeding due to taking painkillers, a prediction model which will be described below may discover and predict that the user has and will have a family medical history of digestive issues. To this end, a large language model (LLM)-based generative AI, such as ChatGPT or GPT 4.0, may be used, and fine-tuning data utilized for the LLM-based generative AI may be public medical data. The online medical questionnaire unit 360 may inquire about the user's family medical history while checking disease codes, diagnostic names, etc. in the user's recent medical records, inquire about the user's medical history over his or her lifetime that is not included in the user's recent medical history, etc, and receive answers thereon.

When the research institute terminal 400 finally selects the user of the user terminal 100 as a clinical target subject and the clinical trial is conducted and completed, the reward management unit 370 may check preset compensation payment conditions and then make the payment to the user of the user terminal 100. For example, participating in a clinical trial involves multiple trips to and from a hospital, which may offer a hassle and inconvenience, and users who comply with all of the trips may be compensated. Here, demographic data of compensated users and uncompensated users may be collected to obtain characteristics of a group of users who have followed all of the rules and remained until the end of the clinical trial and characteristics of those who have not. After characteristics of each group are obtained, it is possible to not only obtain individuals who satisfy clinical trial requirements as clinical trial subjects but also obtain subjects who are likely to complete clinical trials.

TABLE 2
Clinical trial delay factor (IBM Global Business Services survey)

| 1 | Failure to recruit subjects on time (75% of clinical trials) | |
|---|---|---|
| 2 | Failure to maintain | A) Self-relinquishment due to |
| | enrollment within | inconvenience of trips to and |
| | the trial period | from the hospital |
| | (85% of clinical trials) | B) Eliminated for violations |

### <Clustering>

Hierarchical clustering may be performed using a K-means algorithm on the basis of each user's demographic data. In addition to this, various non-supervised learning-based deep learning algorithms, such as mean shift, Gaussian mixture model (GMM), density-based spatial clustering of applications with noise (DBSCAN), etc. may be used. Here, deep-learning algorithms are not limited to those listed above and are not excluded because they have not been listed. After clusters are made to extract features of those who have finished clinical trials and those who have not, if the population is large enough to recruit subjects, subjects may be selected, as described above, by including subjects with the features of those who have completed clinical trials and excluding subjects with the features of those who have not done so.

When a wearable device is used at the user terminal 100 and the user terminal 100 consents to collect and use bio-information data collected from the wearable device, the blank fill-in unit 380 may compare bio-information data measured from the wearable device with the clinical trial requirements to determine whether the bio-information data satisfies the clinical trial requirements. For example, when the clinical trial requirements include information on “recent systolic blood pressure” as shown in FIG. 3J and systolic blood pressure has been collected by the wearable device, the systolic blood pressure may be used. In other words, the recent systolic blood pressure may be used in the case in which medical data includes clinical trial requirements (systolic blood pressure) and recent data does not exist but is necessary, the case in which medical data does not include clinical trial requirements (systolic blood pressure), or other cases.

When the number of targets matching the clinical trial requirements is lower than a preset number, the possibility prediction unit 390 may predict a current health state by inputting medical data into the previously prepared prediction model to increase the number of targets, and may sort a corresponding user into a possibility group and then transmit clinical trial information of the clinical trial to the possibility group when the health state matches the clinical trial requirements. For example, when user Z satisfies clinical trial requirements A, B, and, C but does not satisfy clinical trial requirement D, user Z is excluded, in principle, from the subjects to be recruited. In this case, when there is a possibility that clinical trial requirement D is satisfied, or clinical trial requirement D is predicted but is not known, clinical trial information of a possibility group including user Z may be transmitted. To this end, the disease prediction algorithm may be used.

### <Prediction Model>

Using retinal disease as an example, a multi-layer perceptron (MLP) may be used to predict whether patients will develop a retinal disease on the basis of their disease codes. Alternatively, disease codes may be listed in a history log to generate time-series data, which may be used to predict whether a retinal disease will develop. In other words, disease codes related to retinal diseases are obtained by obtaining correlations on the basis of machine learning, and when disease codes related to retinal diseases are obtained from among disease codes, time-series data is generated using only the related disease codes and whether a retinal disease will develop is predicted by analyzing the time-series data. For example, when there are disease codes A-B-C-D-E, disease codes related to retinal diseases are A-C-D-E among the disease codes, and the process of A-C-D-E leads to a retinal disease, specifically, neovascular age-related macular degeneration (nAMD), a user who is currently going through the process of A-C-D-E may be included in a clinical trial related to macular degeneration because it is possible that the user currently has macular degeneration even though it has not been currently diagnosed.

An operational process according to the above-described configuration of the management service provision server of FIG. 2 will be described in detail below utilizing examples of FIGS. 3 and 4. However, the exemplary embodiment is only one of various embodiments of the present invention, and the present invention is obviously not limited thereto.

Referring to FIG. 3A, (a) the management service provision server 300 may collect clinical trial requirements from the research institute terminal 400, store the collected clinical trial requirements, may map a user to the medical data and store the medical data when the user terminal 100 provides a right to access medical data to check insurance coverage as shown in (b), and then may generate a list of clinical trials for which clinical trial requirements are satisfied based on the user's medical data and transmit the clinical trial list to the user terminal 100 to propose participation in a clinical trial. At this time, (d) when there is application for participation from the user terminal 100, the management service provision server 300 may receive an electronic signature to collect medical history data as shown in (a) of FIG. 3B. When the medical history data satisfies clinical trial requirements, (b) the management service provision server 300 may carry out an online medical questionnaire to obtain the user's responses. When the user is selected as a subject on the basis of the user's responses, the user visits a hospital and has a health checkup to be ultimately examined as shown in (c). (c) shows entities that perform each of the processes and operations of each of the processes. As shown in (d), when the clinical trial is finished and the user satisfies the preset compensation payment conditions, the management service provision server 300 makes the payment to the user terminal 100.

FIG. 3C shows the company (LifeCatch) of the applicant of the present invention, which provides a service as shown in FIG. 3D. While clinical trials are currently conducted as shown in FIGS. 3E and 3F, according to an exemplary embodiment of the present invention, targeting subjects who satisfy clinical trial requirements as shown in FIG. 3G leads to success in microtargeting through a process as shown in FIG. 3H. Accordingly, the number of screened subjects is reduced while the possibility of a user becoming a subject is increased, which saves manpower and time. In addition, it is possible to minimize delays in bringing a new drug to the market due to clinical delays as a result of minimizing recruitment failure or dropouts as shown in Table 2. This is summarized as shown in FIG. 3I, and the platform of the present invention actually completed recruitment of subjects in two days as shown in FIG. 3J. Such a clinical trial generally has a recruitment period of about 4.5 months as shown in FIG. 3K, but the platform of the present invention completed recruitment only in two days as shown in FIG. 3L. The company is currently receiving clinical trial recruitment requests from research institutions as shown in FIG. 3M. Requests for clinical trial recruiting may be induced by simulating how many subjects are recruitable in two days. This simulation may be accomplished by collecting application guidelines (inclusions and exclusions) of a research institution, obtaining users with medical data corresponding to the application guidelines, and transmitting the number of users. This can revolutionize the pharmaceutical and biotechnology industry by reducing recruitment periods, the biggest hurdle in phase 3, by more than 80%. In addition, users will have better access to clinical trial information and can quickly and accurately decide on a clinical trial and then apply for the clinical trial because clinical trials that the users are eligible to participate in have been selected.

Matters not described regarding the method of providing a clinical trial target recruitment management service of FIGS. 2, FIGS. 3A-3M and 4A-4I are identical to or may be readily inferable from matters previously described regarding the method of providing a clinical trial target recruitment management service of FIG. 1 and are therefore omitted from the following description.

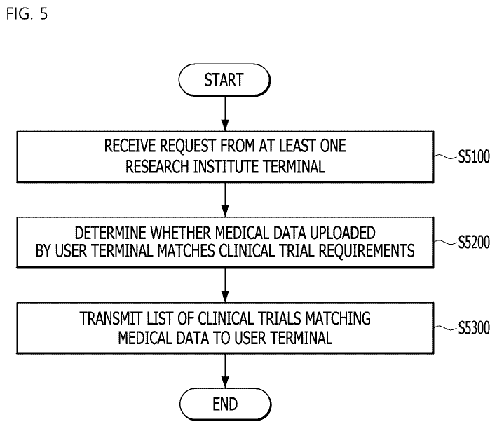

FIG. 5 is a diagram illustrating a process in which data is transmitted and received between elements included in the system for recruiting clinical trial targets on the basis of big data shown in FIG. 1 according to an exemplary embodiment of the present invention. An example of the process in which data is transmitted and received between the elements will be described below with reference to FIG. 5. However, the present invention is not limited thereto, and it is obvious to those of ordinary skill in the art that the data transmission and reception process shown in FIG. 5 may be modified according to the various embodiments described above.

Referring to FIG. 5, the management service provision server receives a request from the at least one research institute terminal (S5100) and determines whether medical data uploaded by the user terminal matches clinical trial requirements (S5200).

The management service provision server transmits a list of clinical trials matching the medical data to the user terminal (S5300).

The order of the above-described operations S5100 to S5300 is illustrative, and the operations S5100 to S5300 are not limited thereto. In other words, the order of the above-described operations S5100 to S5300 may be changed, and some of the operations S5100 to S5300 may be simultaneously performed or omitted.

Matters not described regarding the method of providing a clinical trial target recruitment management service of FIG. 5 are identical to or may be readily inferable from matters previously described regarding the method of providing a clinical trial target recruitment management service in FIGS. 1 and 2, FIGS. 3A-3M and 4A-4I and are therefore omitted from the following description.

The method of providing the clinical trial target recruitment management service according to the exemplary embodiment described with reference to FIG. 5 may be implemented in the form of a recording medium including instructions executable by a computer such as an application or program module executed by a computer. A computer-readable medium may be any available medium that is accessible by a computer and includes both volatile and non-volatile media and removable and non-removable media. In addition, the computer-readable medium may include all types of computer storage media. The computer storage media include both volatile and non-volatile media and removable and non-removable media implemented by any method or technology for storing information such as computer-readable instructions, data structures, program module, or other data.

The above-described method of providing a clinical trial target recruitment management service according to the exemplary embodiment of the present invention may be executed by an application (which may include a program included in a platform, an operating system (OS), etc. installed by default on a terminal) that is installed by default on the terminal and may be executed by an application (i.e., a program) installed directly on a master terminal by a user through an application provision server such as an application store server, a web server related to an application or the service, etc. In this sense, the above-described method of providing a clinical trial target recruitment management service according to the exemplary embodiment may be implemented as an application (i.e., a program) installed by default on a terminal or directly installed by a user and may be recorded on a recording medium that is readable by a computer such as a terminal or the like.

According to any one of the above-described solutions of the present invention, it is possible to store medical data mapped to the user terminal when medical data is uploaded from a user terminal to inquire about available insurance coverage, obtain user terminals having medical data corresponding to the clinical trial requirements when a research institution terminal requests target recruiting while registering clinical trial requirements, propose participation in a clinical trial while transmitting a list of matching clinical trials to the user terminals, and thereby help recruit subjects rapidly and accurately and complete clinical trials without delays.

The above description of the present invention is for illustrative purposes, and those skilled in the technical field to which the present invention pertains will understand that it may be easily modified into other specific forms without changing the technical spirit or essential features of the present invention. Therefore, it is to be understood that the embodiments described above are illustrative rather than restrictive in all aspects. For example, each component described as singular may be implemented in a distributed manner, and similarly, components described as being distributed may be implemented in a combined form.

The scope of the present invention is defined by the claims rather than the above detailed description, and all modifications and alterations derived from the claims and their equivalents should be construed as falling within the scope of the present invention.

What is claimed is:

1. A system for recruiting clinical trial targets on the basis of big data, the system comprising:
a user terminal configured to upload medical data to inquire about available insurance funds and receive a list of clinical trials matching the medical data;
at least one research institution terminal configured to register clinical trial requirements and request recruitment of clinical trial targets; and
a management service provision server including a reception unit configured to receive the request from the at least one research institution terminal, a matching determination unit configured to determine whether the medical data uploaded by the user terminal matches the clinical trial requirements, and a transmission unit configured to transmit a list of clinical trials matching the medical data to the user terminal.

2. The system of claim 1, wherein the management service provision server further includes an electronic signature unit configured to provide an electronic signature link to the user terminal to obtain written consent to apply for participation when an application for participation in at least one clinical trial from the list of clinical trials is provided from the user terminal and map an electronic signature of the user terminal to the user terminal and store the electronic signature when the electronic signature is completed on the user terminal.

3. The system of claim 2, wherein the management service provision server further includes a medical history check unit configured to collect medical history data of the user terminal from at least one information provision server after the consent is obtained.

4. The system of claim 2, wherein the management service provision server further includes an online questionnaire unit configured to provide an online questionnaire to the user terminal to determine whether a user satisfies the clinical trial requirements after the consent is obtained.

5. The system of claim 1, wherein the management service provision server further includes a reward management unit configured to check preset reward payment conditions and then make a payment to a user of the user terminal when the research institute terminal finally selects the user of the user terminal as a clinical target subject and the clinical trial has been conducted and completed.

6. The system of claim 1, wherein the management service provision server further includes a blank fill-out unit configured to compare bio-information data measured from a wearable device with the clinical trial requirements to determine whether the bio-information data satisfies the clinical trial requirements when the wearable device is used at the user terminal and the user terminal consents to collect and use bio-information data collected by the wearable device.

7. The system of claim 1, wherein the management service provision server further includes a possibility prediction unit configured to predict a current health state by inputting the medical data into a previously prepared prediction model to increase a number of targets when the number of targets matching the clinical trial requirements is lower than a preset number, and categorize a user into a possibility group and then transmit clinical trial information of the clinical trial to the possibility group when the health state matches the clinical trial requirements.
